(19) United States
(12) Patent Application Publication
(10) Pub. No.: US 2023/0149282 A1
(43) Pub. Date: May 18, 2023

(54) High-Potency Vitamin C Chemical Peeling Solutions

(71) Applicant: Baek Clinical Inc., Los Angeles, CA (US)

(72) Inventor: Jihoon P. BAEK, Los Angeles, CA (US)

(21) Appl. No.: 17/918,820

(22) PCT Filed: Apr. 16, 2021
(86) PCT No.: PCT/US2021/027834

§ 371 Date: Oct. 13, 2022

Related U.S. Application Data
(60) Provisional application No. 63/010,881, filed on Apr. 16, 2020.

(51) Int. Cl. A61K 8/67; A61K 8/42; A61K 8/368; A61K 8/34; A61K 8/365; A61K 8/06; A61Q 19/08; A61Q 19/02

(52) U.S. Cl. CPC A61K 8/676; A61K 8/42; A61K 8/062; A61K 8/345; A61K 8/365; A61K 8/368; A61Q 19/02; A61Q 19/08; A61K 2800/28; A61K 2800/49; A61K 2800/522; A61K 2800/5922

(57) ABSTRACT

Topical formulations including a combination of L-ascorbic acid, a urea agent and a chemical exfoliant dissolved in a non-aqueous skin-compatible solvent are provided. The topical formulations including a chemical exfoliant can be used as a chemical peeling solution. The formulations are storage stable for an extended period of time without significant degradation of the L-ascorbic acid or urea in the composition, are have several desirable physical properties when topically applied to skin. Topical compositions of this disclosure find use in treating or preventing a variety of cosmetic and/or dermatological conditions as well as to reduce the appearance of chronological and/or environmentally-caused skin aging. In some embodiments, the topical composition finds use as a chemical peeling solution, e.g., to facilitate shedding of the top layers of skin cells after topical administration.

### CROSS-REFERENCE TO RELATED APPLICATIONS

This application claims the benefit of U.S. Provisional Application No. 63/010,881, filed Apr. 16, 2020, the disclosure of which is hereby incorporated by reference in its entirety.

### FIELD OF INVENTION

Compositions and methods for treating, preventing, or improving dermatocosmetic conditions, including reducing the appearance of skin aging.

### INTRODUCTION

Ascorbic acid (also commonly known as Vitamin C) is a potent antioxidant and is widely used in topical compositions to treat or prevent a range of cosmetic and/or dermatological conditions as well as to reduce the appearance of chronological and/or environmentally-caused skin aging, such as facial fine lines and wrinkles, dyschromia/uneven pigmentation, and dark circles under the eyes). Additionally, Vitamin C can help neutralize the damaging effects of free radicals and plays a role in stimulating the growth and bundling of collagen, important in maintaining skin elasticity. Tyrosinase is a copper-containing enzyme that catalyzes the production of melanin and other pigments from tyrosine by oxidation. The antioxidant activity of ascorbic acid is reported to mediate, and thereby reduce (inhibit) the rate of melanogenesis. YK Choi et al, Int J Dermatol. Vol. 49, pp. 218-26 (2010).

The “gold standard” in cosmetic dermatology for skin lightening/brightening is hydroquinone (HQ). However, HQ can have side effects including mild burning, stinging, erythema (redness), and skin dryness. Vitamin C is also used to lighten the appearance of the skin - including for example, dark circles under the eyes - but with a more favorable safety profile (i.e., fewer side effects). See, e.g., LE Espinal-Perez et al, Int J Dermatol. Vol. 43, pp. 604-7 (2004) (93% improvement from use of 4% HQ versus 62.5% improvement from use of 5% Vitamin C; but 68.7% side-effects from HQ versus 6.2% from Vitamin C).

The scientific and patent literature describe Vitamin C topical products, especially water-containing formulations, as “unstable”. Research and development activities seeking more stable topical Vitamin C formulations have focused on creating esterified derivatives (e.g., magnesium ascorbyl phosphate (“MAP”) and ascorbyl-6-palmitate), using anhydrous carrier systems, adding antioxidants or other ingredients to Vitamin C formulations, and buffering Vitamin C formulations to a low pH.

The efficacy of Vitamin C formulations depends to a large extent on concentration. For example, a cream containing 10% MAP is reported to effectively brighten/lighten the appearance of the skin. See K Kameyama et al. J Am. Acad. Dermatol. Vol. 34, pp. 29-33 (1996). However, many skin care products contain vitamin C or a derivative at concentrations of less than 1%. R. Sarkar et al. J Cutan Aesthet. Surg. Vol. 6, No. 1, pp. 4-11 (2013).

Researchers in the Department of Chemical and Biomolecular Engineering, Yonsei University, Seoul, Republic of Korea investigated carrier-based approaches for reducing the oxidation of L-ascorbic acid in cosmetic emulsions. Emulsion stability (i.e., not separating into oil and water phases) as well as the effects of changes in the pH, color, and concentration of L-ascorbic acid were studied in four types of emulsions: water-in-oil (W/O), propylene glycol-in-oil (PG/O), butylene glycol-in-oil (B/O), and glycerine-in-oil (G/O) emulsions. The G/O emulsion that used glycerine as the dispersed phase retained the highest proportion of the initial Z-ascorbic acid (AA) content over time, followed by the PG/O, B/O, and W/O emulsions. Sehui Kim, Tai Gyu Lee “Stabilization of Z-ascorbic acid in cosmetic emulsions” J. Ind. Chem. Eng. Vol. 57, pp. 193-198 (2018).

In topical compositions, the use of urea (and substituted ureas) is known, including for moisture retention (as a humectant), for keratolytic activity, as well as for penetration enhancement, both for itself and other active ingredients. At concentrations of lower than about 10%, urea acts as a moisturizer. At higher concentrations, from about 10% up to 40%, urea can be used to treat dry/rough skin conditions, including ichthyosis and psoriasis.

It is also known in the art that inclusion of urea at efficacious concentrations in aqueous topical compositions poses formulating challenges. Urea undergoes steady hydrolysis, producing ammonia and other amines, compounds that not only have an unpleasant odor but also tend to increase pH. Moreover, hydrolysis of urea in aqueous compositions can cause discoloration or other breakdown of the product, including phase separation.

There has been and remains a need for non-oily/non-greasy topical formulations that contain and maintain a high concentration of Vitamin C and efficacious amounts of urea without degradation, and concomitant decrease in biological activity. These needs are met by the high-potency Vitamin C concentrates of the present disclosure.

### SUMMARY

Topical formulations including a combination of L-ascorbic acid, a urea agent and a chemical exfoliant dissolved in a non-aqueous skin-compatible solvent are provided. The topical formulations including a chemical exfoliant can be used as a chemical peeling solution. The formulations are storage stable for an extended period of time without significant degradation of the Z-ascorbic acid or urea in the composition, are have several desirable physical properties when topically applied to skin. The compositions of this disclosure are stable liquid compositions with respect to both the ascorbic acid component and the urea component. The topical formulations can include high concentrations of the Z-ascorbic acid of 10 to 28% by weight. Topical compositions of this disclosure find use in treating or preventing a variety of cosmetic and/or dermatological conditions as well as to reduce the appearance of chronological and/or environmentally-caused skin aging. In some embodiments, the topical composition finds use as a chemical peeling solution, e.g., to facilitate shedding of the top layers of skin cells after topical administration.

### DETAILED DESCRIPTION

This disclosure provides topical formulations of L-ascorbic acid dissolved in a combination of a urea agent and a non-aqueous skin-compatible solvent. The formulations are storage stable for an extended period of time without undesirable discoloration or significant degradation of the Z-ascorbic acid in the composition. This disclosure provides particular topical formulations which have been developed and optimized to provide skin compatibility and desirable physical properties.

Topical compositions of this disclosure find use in treating or preventing a variety of cosmetic and/or dermatological conditions as well as to reduce the appearance of chronological and/or environmentally-caused skin aging, such as facial fine lines and wrinkles, dyschromia or uneven pigmentation, and dark circles under the eyes. Non-limiting examples of dermatocosmetic conditions that may be improved by topical application of the compositions of the present disclosure include: keratoses, melasma, lentigines, liver spots, inflammatory dermatoses (including eczema, acne, psoriasis), and xeroses (also known in the art as dry skin or pruritus).

In some embodiments, formulations of the present disclosure include the ingredients: (i) 5 to 28 % by weight ascorbic acid; and (ii) urea agent; dissolved in (iii) a non-aqueous skin-compatible solvent.

### Ascorbic Acid

This disclosure provides formulations that include combination of particular amount of a urea agent in a non-aqueous skin-compatible solvent which together can provide for dissolution of particular amounts of ascorbic acid and which produce skin-compatible liquid compositions in which the ascorbic acid is substantially stable to decomposition. In some embodiments, the amounts of ascorbic acid stably dissolved in the composition are greater than would otherwise be possible without the particular combinations of ingredients provided by theirs disclosure.

The terms “ascorbic acid”, “L-ascorbic acid” and “vitamin C” are used interchangeably herein, and refer to the naturally occurring vitamin of CAS Registry Number: 50-81-7. Any convenient form of ascorbic acid can be utilized in the subject formulations. In some embodiments, the ascorbic acid used in the high potency Vitamin C concentrate of the present disclosure is a powder.

In certain embodiments, the ascorbic acid material used in preparing the subject compositions is composed of granular particles. Such a particulate powder has a particle size (e.g., mean particle size) of less than about 25 microns, such as less than about 20 microns, and more preferably less than about 12.5 microns, e.g., as measured by a Hagman gauge. In some embodiments, all of the ascorbic acid powder used in preparing the subject compositions is capable of passage through a No. 100 U.S. Standard Sieve, a standard testing procedure used by the US Pharmacopoeia. In some embodiments, 80% or more (such as 90% or more, or 100%) of ascorbic acid powder used in preparing the subject composition is capable of passage through a No. 325 U.S. Standard Sieve. For example, one powder meeting the above criterion is Ascorbic Acid Ultra-Fine Powder from DSM Nutritional Products LLC, Parsippany, NJ. Previously, this product was available as Product Code No. 6045653 from Roche Vitamins and Fine Chemicals.

In some embodiments, the amount of ascorbic acid in the subject composition is at least about 5% by weight, such as at least about 10% by weight, at least about 12% by weight, at least about 15% by weight, at least about 20% by weight, or at least about 25% by weight. In some embodiments, the subject composition includes about 28% by weight or less of ascorbic acid in the non-aqueous solvent solution, such as about 25% by weight or less. In certain embodiments, the non-aqueous solvent is 1,3-propanediol. In particular embodiments, the amount of ascorbic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of ascorbic acid in the subject composition is about 5%, about 10%, about 15%, about 20%, or about 25% by weight.

In some embodiments, the amount of ascorbic acid in the subject composition is about 5%, about 6%, about 7%, about 8%, about 9%, about 10%, about 11%, about 12%, about 13%, about 14%, about 16%, about 17%, about 18%, about 19%, about 20%, about 21%, about 22%, about 23%, about 24%, about 25%, about 26%, about 27%, about 28%, about 29%, or about 30% by weight.

In particular embodiments, the amount of ascorbic acid in the subject composition is between about 10% by weight and about 20% by weight (e.g., about 10%, about 15%, or about 20%) where the ratio of ascorbic acid to urea agent (% wt ratio) is 1.8 to 2.2, such as a ratio of 2 (i.e., 2:1).

In particular embodiments, the amount of ascorbic acid in the subject composition is between about 25% by weight and about 28% by weight (e.g., about 25%, about 26%, about 27% or about 28%) where the ratio of ascorbic acid to urea agent (% wt ratio) is 1.0 to 1.3, such as a ratio of 1.25 (i.e., 1.25:1) or a ratio of 1.0 (i.e., 1:1).

In general, the amounts of ascorbic acid in a composition are calculated relative to the solution phase based on the non-aqueous solvent. See Formulations 1, 3, 4, 6 and 7 of Table 2. However, the amounts of ascorbic acid and other ingredients relative to the emulsion composition as a whole can readily be calculated by the skilled artisan. Formulations 2 and 5 of Table 2 show exemplary emulsion compositions where the % by weight values shown are relative to the total emulsion composition. It is understood that, in some cases, these concentrate solutions having particular amounts of ascorbic acid can be combined with an immiscible ingredient (e.g., a oil component) and an emulsifying agent to produce an emulsion composition (e.g., as described below).

In some embodiments, the ascorbic acid is dissolved at a concentration [AA] that is above its maximum concentration in the solvent alone [X], and the urea is dissolved at a concentration that is at least ([AA]-[X])*1.25.

### Urea Agent

The formulations of the present disclosure include a urea agent in an amount sufficient to enhance the solubility of ascorbic acid in the non-aqueous skin compatible solvent and to provide a stable solution. The inventor discovered that particular amounts of urea agent can be added to a non-aqueous solvent to provide stable solutions of ascorbic acid at various desired concentration levels. These amounts of urea agent are selected based on observations regarding the maximum amount of ascorbic acid that can be stably dissolved in the particular non-aqueous solvent, and minimum amounts of urea agent that should be included to provide a stable ascorbic acid solution.

Urea agents of interest include, but are not limited to, urea and substituted urea, such as alkyl substituted urea, more particularly mono-substituted or di-substituted alkyl urea (e.g., hydroxyalkyl urea). In some embodiments, the urea agent is a hydroxyalkyl urea, such as hydroxyethyl urea. The urea agent ingredient used in the subject formulations can be a combination of urea and/or substituted ureas. For example, the urea agent can be a combination of urea and hydroxyethyl urea. In certain embodiments, the urea agent is urea. In certain embodiments, the urea agent is hydroxyethyl urea.

In some embodiments, the amount of urea in the high-potency vitamin C compositions of this disclosure is defined as a function of the concentration of L-Ascorbic Acid (“AA”). For AA concentrations exceeding the maximum solubility of ascorbic acid in the neat non-aqueous solvent (Z%), as a first step, subtract Z from the desired concentration of AA in the solution. As a second step, multiply the difference from the first step by 1.25. The minimum amount (% wt) of urea agent to be included in the non-aqueous solvent based compositions can be calculated by the formula: {concentration of AA - Z} * 1.25.

For compositions based on 1,3-propanediol as solvent, the maximum solubility of ascorbic acid (AA) in neat 1,3-propanediol was observed to be 12% by weight. Accordingly, for AA concentrations exceeding 12%, as a first step, subtract 12 from the desired amount of AA in the concentrate. As a second step, multiply the difference from the first step by 1.25. The minimum amount (% wt) of urea agent to be included in the 1,3-propanediol based compositions can be calculated by the formula: {concentration of AA - 12} * 1.25. See Table 1.

TABLE 1

Minimum urea agent in 1,3-propanediol compositions {concentration of AA - 12} * 1.25

| ascorbic acid (% wt) | Minimum urea agent (% wt) |
|---|---|
| 13 | 1.25 |
| 14 | 2.5 |
| 15 | 3.75 |
| 16 | 5 |
| 17 | 6.25 |
| 18 | 7.5 |
| 19 | 8.75 |
| 20 | 10 |
| 21 | 11.25 |
| 22 | 12.5 |
| 23 | 13.75 |
| 24 | 15 |
| 25 | 16.25 |
| 26 | 17.5 |
| 27 | 18.75 |
| 28 | 20 |

In some embodiments, the ascorbic acid is dissolved at a concentration [AA] that is above its maximum concentration in the solvent alone [X], and the urea is dissolved at a concentration that is at least ([AA]-[X])* 1.25.

For example, for compositions including 15% by weight ascorbic acid, at least about 4% urea is included in the 1,3-propanediol solvent. For compositions including 20% by weight ascorbic acid, at least about 10% urea is included in the 1,3-propanediol solvent. For compositions including 25% by weight ascorbic acid, at least about 16% urea is included in the 1,3-propanediol solvent. In some embodiments, additional amounts of urea agent can be included up to a maximum amount of 20% by weight, to provide desirable physical properties, in combination with additional optional minor ingredients.

In some embodiments, the subject composition includes about 13 to 19% by weight ascorbic acid, about 2 to about 9% by weight urea agent and 1,3-propanediol. In some embodiments, the subject composition includes about 15% by weight ascorbic acid, about 2 to about 9% by weight urea agent (e.g., about 4%, about 5%, about 6%, about 7% or about 8%) and 1,3-propanediol. In certain embodiments, the subject composition includes about 15% by weight ascorbic acid, about 8% by weight urea agent and 1,3-propanediol.

In some embodiments, the subject composition includes about 20 to 24% by weight ascorbic acid, about 10 to about 15% by weight urea agent and 1,3-propanediol. In some embodiments, the subject composition includes about 20% by weight ascorbic acid, about 10 to about 15% by weight urea agent (e.g., about 10%, about 11%, about 12%, about 13%, about 14% or about 15%) and 1,3-propanediol. In certain embodiments, the subject composition includes about 20% by weight ascorbic acid, about 10% by weight urea agent and 1,3-propanediol.

In some embodiments, the subject composition includes about 25 to 28% by weight ascorbic acid, about 16 to about 20% by weight urea agent and 1,3-propanediol. In some embodiments, the subject composition includes about 25% by weight ascorbic acid, about 16 to about 20% by weight urea agent (e.g., about 16%, about 17%, about 18%, about 19%, or about 20%) and 1,3-propanediol. In certain embodiments, the subject composition includes about 25% by weight ascorbic acid, about 20% by weight urea agent and 1,3-propanediol.

### Chemical Exfoliant

The formulations of the present disclosure include a chemical exfoliant. A chemical exfoliant is an agent capable of facilitating the shed of top layers of skin cells. The chemical exfoliant can be a small organic molecule that includes a carboxylic acid group and a hydroxy group. This disclosure provides topical compositions that include a combination of ascorbic acid, urea agent and a chemical exfoliant dissolved in a non-aqueous solvent. The compositions of this disclosure are stable liquid compositions with respect to both the ascorbic acid component and the urea component.

In some embodiments, the chemical exfoliant is an alpha hydroxy acid or a beta hydroxy acid. The acid may be an alkyl carboxylic acid or a benzoic acid (e.g., a hydroxy-substituted benzoic acid). The hydroxy group can be a phenol or an alkyl alcohol. In certain embodiments, the chemical exfoliant is an alpha-hydroxy carboxylic acid. In certain embodiments the chemical exfoliant contains 2-12 carbon atoms, such as 2-6 or 2-4 carbons. Chemical exfoliants of interest include, but are not limited to, glycolic acid, lactic acid, mandelic acid, salicylic acid, capryloyl salicylic acid, salicyloyl phytosphingosine, phenol, gluconolactone, lactobionic acid, maltobionic acid, and combinations thereof. In some embodiments, the chemical exfoliant is salicylic acid.

In some embodiments, the amount of the chemical exfoliant in the subject composition ranges from 2% to 50% by weight of a chemical exfoliant, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of a chemical exfoliant. In some embodiments, the amount of the chemical exfoliant in the subject composition ranges from 2% to 30% by weight.

In some embodiments, the amount of chemical exfoliant in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of a chemical exfoliant in the non-aqueous solvent solution, such as about 25% by weight or less. In certain embodiments, the non-aqueous solvent is 1,3-propanediol. In particular embodiments, the amount of chemical exfoliant in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of chemical exfoliant in the subject composition is about 2%, about 5%, about 10%, about 15%, about 20%, about 25%, about 30%, about 35%, about 40%, about 45%, or about 50% by weight.

In some embodiments, the chemical exfoliant is glycolic acid. In some embodiments, the amount of glycolic acid in the subject composition ranges from 2% to 50% by weight of glycolic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of glycolic acid.

In some embodiments, the amount of glycolic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of glycolic acid in the non-aqueous solvent solution, such as about 25% by weight or less. In certain embodiments, the non-aqueous solvent is 1,3-propanediol. In particular embodiments, the amount of glycolic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of glycolic acid in the subject composition is about 2%, about 5%, about 10%, about 15%, about 20%, about 25%, about 30%, about 35%, about 40%, about 45%, or about 50% by weight.

In some embodiments, the chemical exfoliant is lactic acid. In some embodiments, the amount of lactic acid in the subject composition ranges from 2% to 50% by weight of lactic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of lactic acid.

In some embodiments, the amount of lactic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of lactic acid in the non-aqueous solvent solution, such as about 25% by weight or less. In certain embodiments, the non-aqueous solvent is 1,3-propanediol. In particular embodiments, the amount of lactic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of lactic acid in the subject composition is about 2%, about 5%, about 10%, about 15%, about 20%, about 25%, about 30%, about 35%, about 40%, about 45%, or about 50% by weight.

In some embodiments, the chemical exfoliant is mandelic acid. In some embodiments, the amount of mandelic acid in the subject composition ranges from 2% to 50% by weight of mandelic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of mandelic acid.

In some embodiments, the amount of mandelic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of mandelic acid in the non-aqueous solvent solution, such as about 25% by weight or less. In certain embodiments, the non-aqueous solvent is 1,3-propanediol. In particular embodiments, the amount of mandelic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of mandelic acid in the subject composition is about 2%, about 5%, about 10%, about 15%, about 20%, about 25%, about 30%, about 35%, about 40%, about 45%, or about 50% by weight.

In some embodiments, the chemical exfoliant is salicylic acid. In some embodiments, the amount of salicylic acid in the subject composition ranges from 2% to 50% by weight of salicylic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of salicylic acid.

In some embodiments, the amount of salicylic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of salicylic acid in the non-aqueous solvent solution, such as about 25% by weight or less. In certain embodiments, the non-aqueous solvent is 1,3-propanediol. In particular embodiments, the amount of salicylic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of salicylic acid in the subject composition is about 2%, about 5%, about 10%, about 15%, about 20%, about 25%, about 30%, about 35%, about 40%, about 45%, or about 50% by weight.

In some embodiments, the chemical exfoliant is capryloyl salicylic acid. In some embodiments, the amount of capryloyl salicylic acid in the subject composition ranges from 2% to 50% by weight of capryloyl salicylic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of capryloyl salicylic acid.

In some embodiments, the amount of capryloyl salicylic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of capryloyl salicylic acid in the non-aqueous solvent solution, such as about 25% by weight or less. In certain embodiments, the non-aqueous solvent is 1,3-propanediol. In particular embodiments, the amount of capryloyl salicylic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of capryloyl salicylic acid in the subject composition is about 2%, about 5%, about 10%, about 15%, about 20%, about 25%, about 30%, about 35%, about 40%, about 45%, or about 50% by weight.

In some embodiments, the chemical exfoliant is salicyloyl phytosphingosine. In some embodiments, the amount of salicyloyl phytosphingosine in the subject composition ranges from 2% to 50% by weight of salicyloyl phytosphingosine, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of salicyloyl phytosphingosine.

In some embodiments, the amount of salicyloyl phytosphingosine in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of salicyloyl phytosphingosine in the non-aqueous solvent solution, such as about 25% by weight or less. In certain embodiments, the non-aqueous solvent is 1,3-propanediol. In particular embodiments, the amount of salicyloyl phytosphingosine in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of salicyloyl phytosphingosine in the subject composition is about 2%, about 5%, about 10%, about 15%, about 20%, about 25%, about 30%, about 35%, about 40%, about 45%, or about 50% by weight.

In some embodiments, the chemical exfoliant is phenol. In some embodiments, the amount of phenol in the subject composition ranges from 2% to 50% by weight of phenol, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of phenol.

In some embodiments, the amount of phenol in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of phenol in the non-aqueous solvent solution, such as about 25% by weight or less. In certain embodiments, the non-aqueous solvent is 1,3-propanediol. In particular embodiments, the amount of phenol in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of phenol in the subject composition is about 2%, about 5%, about 10%, about 15%, about 20%, about 25%, about 30%, about 35%, about 40%, about 45%, or about 50% by weight.

In some embodiments, the chemical exfoliant is gluconolactone. In some embodiments, the amount of gluconolactone in the subject composition ranges from 2% to 50% by weight of gluconolactone, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of gluconolactone.

In some embodiments, the amount of gluconolactone in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of gluconolactone in the non-aqueous solvent solution, such as about 25% by weight or less. In certain embodiments, the non-aqueous solvent is 1,3-propanediol. In particular embodiments, the amount of gluconolactone in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of gluconolactone in the subject composition is about 2%, about 5%, about 10%, about 15%, about 20%, about 25%, about 30%, about 35%, about 40%, about 45%, or about 50% by weight.

In some embodiments, the chemical exfoliant is lactobionic acid. In some embodiments, the amount of lactobionic acid in the subject composition ranges from 2% to 50% by weight of lactobionic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of lactobionic acid.

In some embodiments, the amount of lactobionic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of lactobionic acid in the non-aqueous solvent solution, such as about 25% by weight or less. In certain embodiments, the non-aqueous solvent is 1,3-propanediol. In particular embodiments, the amount of lactobionic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of lactobionic acid in the subject composition is about 2%, about 5%, about 10%, about 15%, about 20%, about 25%, about 30%, about 35%, about 40%, about 45%, or about 50% by weight.

In some embodiments, the chemical exfoliant is maltobionic acid. In some embodiments, the amount of maltobionic acid in the subject composition ranges from 2% to 50% by weight of maltobionic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of maltobionic acid.

In some embodiments, the amount of maltobionic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of maltobionic acid in the non-aqueous solvent solution, such as about 25% by weight or less. In certain embodiments, the non-aqueous solvent is 1,3-propanediol. In particular embodiments, the amount of maltobionic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of maltobionic acid in the subject composition is about 2%, about 5%, about 10%, about 15%, about 20%, about 25%, about 30%, about 35%, about 40%, about 45%, or about 50% by weight.

In some embodiments, the chemical exfoliant is a combination of two or more of glycolic acid, lactic acid, mandelic acid, salicylic acid, capryloyl salicylic acid, salicyloyl phytosphingosine, phenol, gluconolactone, lactobionic acid, and maltobionic acid. In some embodiments, the amount of two or more of glycolic acid, lactic acid, mandelic acid, salicylic acid, capryloyl salicylic acid, salicyloyl phytosphingosine, phenol, gluconolactone, lactobionic acid, and maltobionic acid in the subject composition ranges from 2% to 50% by weight of two or more of glycolic acid, lactic acid, mandelic acid, salicylic acid, capryloyl salicylic acid, salicyloyl phytosphingosine, phenol, gluconolactone, lactobionic acid, and maltobionic acid, such as 2% to 3%, 3% to 4%, 4% to 5%, 2% to 5%, 5% to 10%, 5% to 15%, 15% to 20%, 20% to 25%, 25% to 30%, 30% to 35%, 35% to 40%, 40% to 45%, or 45% to 50% by weight of two or more of glycolic acid, lactic acid, mandelic acid, salicylic acid, capryloyl salicylic acid, salicyloyl phytosphingosine, phenol, gluconolactone, lactobionic acid, and maltobionic acid.

In some embodiments, the amount of two or more of glycolic acid, lactic acid, mandelic acid, salicylic acid, capryloyl salicylic acid, salicyloyl phytosphingosine, phenol, gluconolactone, lactobionic acid, and maltobionic acid in the subject composition is at least about 2% by weight, such as at least about 3% by weight, at least about 4% by weight, at least about 5% by weight, at least about 10% by weight, at least about 12% by weight, at least about 15% by weight, at least about 20% by weight, at least about 25% by weight, at least about 30% by weight, at least about 35% by weight, at least about 40% by weight, at least about 45% by weight, or at least about 50% by weight. In some embodiments, the subject composition includes about 2% by weight or less of two or more of glycolic acid, lactic acid, mandelic acid, salicylic acid, capryloyl salicylic acid, salicyloyl phytosphingosine, phenol, gluconolactone, lactobionic acid, and maltobionic acid in the non-aqueous solvent solution, such as about 25% by weight or less. In certain embodiments, the non-aqueous solvent is 1,3-propanediol. In particular embodiments, the amount of two or more of glycolic acid, lactic acid, mandelic acid, salicylic acid, capryloyl salicylic acid, salicyloyl phytosphingosine, phenol, gluconolactone, lactobionic acid, and maltobionic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of two or more of glycolic acid, lactic acid, mandelic acid, salicylic acid, capryloyl salicylic acid, salicyloyl phytosphingosine, phenol, gluconolactone, lactobionic acid, and maltobionic acid in the subject composition is about 2%, about 5%, about 10%, about 15%, about 20%, about 25%, about 30%, about 35%, about 40%, about 45%, or about 50% by weight.

### Skin Compatible Solvent

In addition to the urea agent (e.g., as described herein), the high-potency Vitamin C formulations of the present disclosure contain, as an essential ingredient, at least one non-aqueous skin-compatible solvent. A skin compatible solvent is a solvent that does not cause irritation or sensitization when applied topically to the skin. In some embodiments of the chemical peeling solutions of this disclosure, the skin compatible solvent has skin penetration enhancer activity. Any convenient solvents that are penetration enhancers can be adapted for use in the chemical peeling solutions of this disclosure.

Non-aqueous skin-compatible solvents of interest include polyols, C(2-6) alkanediols, glycol ethers, dimethyl ethers, ethanol, isopropyl alcohol, and combinations thereof. In some embodiments, the solvent is a skin compatible polyol. A polyol is an organic alcohol solvent having two or more hydroxy groups. In some embodiments, the polyol solvent is a C(3-6)polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. In some embodiments, the solvent is a mixture of 1,3 propanediol and 1,2 hexanediol.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments of the chemical peeling solutions of this disclosure, the skin compatible solvent comprises ethanol and/or isopropyl alcohol. In some embodiments, the skin compatible solvent comprises ethanol in combination with one or more other solvents (e.g., as described herein). In some embodiments, the skin compatible solvent comprises isopropyl alcohol in combination with one or more other solvents (e.g., as described herein). In some embodiments, the skin compatible solvent itself is composed of more than 50% ethanol and/or isopropyl alcohol, and can provide for enhanced skin penetration of one or more of the other components (e.g., chemical exfoliant) of the chemical peeling solution.

### Additional Components

A formulation may contain one or more (optional) additional ingredients. Any convenient ingredient known to the skilled artisan to provide cosmetic/aesthetic benefits can be utilized in the subject formulations. Such cosmetic/aesthetic benefits include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “glow″/radiance (also described in the art as “brightness”).

In some embodiments, the composition further includes one or more optional additional components (e.g., as described herein). In some embodiments, the one or more optional additional components are selected from tocopherols, tocotrienols (e.g., alpha, beta, delta and gamma tocopherols or alpha, beta, delta and gamma tocotrienols), ferulic acid, azelaic acid, panthenol, pinus pinaster bark extract, emulsifying agent, hyaluronic acid complex, madecassoside, madecassoside asiaticoside, acetyl zingerone, bakuchiol, and bis-ethylhexyl hydroxydimethoxy benzylmalonate.

Each optional additional component (e.g., as described herein) may be present in an amount of 10% or less by weight of the composition, such as 9% or less, 8% or less, 7% or less, 6% or less, 5% or less, 4% or less, 3% or less, 2% or less, 1% or less by weight. In some embodiments the total amount of the one or more optional additional components (e.g., as described herein) in the composition 10% or less by weight, such as 9% or less, 8% or less, 7% or less, 6% or less, 5% or less, 4% or less, 3% or less, 2% or less, 1% or less by weight.

In some embodiments, the composition further includes 10% or less by weight in total of one or more optional additional components selected from an antioxidant, a skin lightening agent, and a moisturizing agent.

### Tocopherol or Tocotrienol Agent

In some embodiments, the composition further includes optional additional component that is a tocopherol or tocotrienol agent. In some embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol.

In some embodiments, the tocopherol or tocotrienol agent is present in the composition in an amount of 2% or less by weight, such as 1.5% or less, 1% or less, or 0.5% or less by weight.

In some embodiments of any one of the formulations described herein, the formulation excludes tocopherol or tocotrienol agents, e.g., or precursors thereof having vitamin E activity. In certain embodiments of any one of the formulations described herein, the formulation excludes vitamin E acetate.

### Antioxidants

In certain embodiments, the formulation contains a secondary antioxidant (i.e., in addition to Vitamin C or the optional additive tocopherol or tocotrienol agent).

Preferred secondary antioxidants include cinnamic acid derivatives (e.g., ferulic acid, caffeic acid, or coumaric acid), terpenoid antioxidants, and benzoic acid derivatives (e.g., p-hydroxy benzoic acid, gallic acid, or protocatechuic acid). Pinus Pinaster Bark/Bud Extract (available under the tradename Pycnogenol® from DKSH North America, Inc., or from Res Pharma Industriale under the tradename Pantrofina® Skin360) contains these cinnamic acid derivatives and benzoic acid derivatives, and is, therefore, a preferred secondary antioxidant.

In some embodiments, the secondary antioxidant is zingerone or acetyl zingerone. In some embodiments, the secondary antioxidant is bakuchiol (10309-37-2) a natural terpenoid antioxidant. In some embodiments, the secondary antioxidant is bis-ethylhexyl hydroxydimethoxy benzylmalonate (HDBM).

The secondary antioxidant, when included, is preferably present in an amount in the range of 0.1 to 3%, more preferably 0.1 to 2% by weight of the composition, such as 0.1 to 1% by weight, 0.1 to 0.5% by weight, e.g., about 0.2%, about 0.3%, about 0.4% or about 0.5% by weight. In some embodiments, the secondary antioxidant is acetyl zingerone.

### Skin Lightening Agents

In certain embodiments, the formulation contains a secondary skin lightening agent (e.g., as defined herein) (i.e., in addition to Vitamin C). Skin lightening agents which may be included in compositions of the present disclosure include, but are not limited to: hydroquinone and its derivatives, including, for example, its monomethyl and monobenzyl ethers; licorice root (Glycyrrhiza glabra) extract; azelaic acid; kojic acid; arbutin; retinoids (including all-trans-retinoic acid, adapalene and tazarotene); gentisic acid (2,5-dihydrobenzoic acid); 4-hydroxy benzoic acid; salts and esters of the above-mentioned acids, including ammonium lactate and sodium lactate; N-acetyl glucosamine; aloesin, a hydroxymethyl chromone isolated from aloe vera; Vitamin B3 compound or its derivative - niacin, nicotinic acid, niacinamide. Epigallocatechin 3-O-gallate (EGCG), and other catechin constituents of tea extracts, in particular green tea; extract of soybean oil (Glycine soja), including isoflavones; hydroxystilbene; butyl hydroxy anisole; and butyl hydroxy toluene may also be utilized as a skin lightening agent. In some embodiments, the additional skin lightening agent is azelaic acid or arbutin.

The skin lightening agent, when included, is preferably present in an amount in the range of 0.1 to 10%, more preferably 0.2 to 5% by weight of the composition, such as 0.2 to 4% by weight, 0.2 to 3% by weight, or 0.2 to 2% by weight. In certain embodiments, the secondary skin lightening agent is soluble and may be added directly to the high Vitamin C (>15%) concentrate of the present invention. The secondary skin lightening agent may also be encapsulated using techniques known to the person having ordinary skill in the art.

### Anti-Inflammatory

In some embodiments, formulation contains an anti-inflammatory agent as an additional ingredient. In some embodiments, the anti-inflammatory agent is madecassoside, madecassoside asiaticoside, or madecassic acid. The anti-inflammatory agent, when included, is preferably present in an amount in the range of 0.1 to 2%, more preferably 0.1 to 1% by weight of the composition, such as 0.1 to 0.5% by weight, or 0.1 to 0.2% by weight. In some embodiments, madecassoside or madecassoside asiaticoside is included in an amount in the range of 0.1 to 0.5%, such as about 0.1% or about 0.2% by weight.

### Storage Stability

High-potency Vitamin C formulations of the present disclosure are capable of maintaining at least 90% of the starting ascorbic acid content when the concentrate is stored at room temperature for 12 months or longer.

The amount of ascorbic acid content in a composition can be determined using a wide range of techniques including, but not limited to: titrimetric, spectrophotometric, electrochemical, fluorimetric, enzymatic and chromatographic. Methods for determining ascorbic acid content in a topical formulation can be complicated/confounded by the presence of excipients or other antioxidant agents (e.g., agents for stabilizing Vitamin C), as well as degradation products. Of the above-listed methods, high performance liquid chromatography is preferred. See, AM Maia et al., “Validation of HPLC stability-indicating method for Vitamin C in semisolid pharmaceutical/ cosmetic preparations ...” Talanta Vol. 71, pp. 639-643 (2007).

In some embodiments, the storage stable composition of this disclosure demonstrates less than 10 mol % degradation of the ascorbic acid after storage for 6 weeks or longer (e.g., 8 weeks or longer, 10 weeks or longer, 12 weeks or longer, 18 weeks or longer, 24 weeks or longer, or even longer) at 40° C. ± 2° C. in a sealed container, such as less than 9 mol %, less than 8 mol %, less than 7 mol %, less than 6 mol %, less than 5 mol %, less than 4 mol %, less than 3 mol %, less than 2 mol % degradation of the ascorbic acid initially present in the composition prior to storage.

In some embodiments, the storage stable composition of this disclosure demonstrates less than 10 mol % degradation of the ascorbic acid after storage for 4 weeks or longer (e.g., 6 weeks or longer, 8 weeks or longer, 10 weeks or longer, 12 weeks or longer, 18 weeks or longer, 24 weeks or longer, or even longer) at 45° C. ± 2° C. in a sealed container, such as less than 9 mol %, less than 8 mol %, less than 7 mol %, less than 6 mol %, less than 5 mol %, less than 4 mol %, less than 3 mol %, less than 2 mol % degradation of the ascorbic acid initially present in the composition prior to storage.

In some embodiments, the storage stable composition of this disclosure demonstrates less than 10 mol % degradation of the ascorbic acid after storage for 6 months or longer (e.g., 8 months or longer, 10 months or longer, 12 months or longer, 18 months or longer, or even longer) at 25° C. ± 2° C. in a sealed container or a multi-use container, such as less than 9 mol %, less than 8 mol %, less than 7 mol %, less than 6 mol %, less than 5 mol %, less than 4 mol %, less than 3 mol %, less than 2 mol % degradation of the ascorbic acid initially present in the composition prior to storage. In certain embodiments, the composition is stored in a sealed container. In certain embodiments, the composition is stored in a multi-use container.

In some embodiments, the storage stable composition of this disclosure demonstrates less than 20 mol % degradation of the ascorbic acid after storage for 12 months or longer (e.g., 18 months or longer, 24 months or longer, or even longer) at 25° C. ± 2° C. in a sealed container or a multi-use container, such as less than 15 mol %, less than 12 mol %, less than 10 mol %, less than 8 mol %, less than 6 mol %, less than 6 mol %, less than 4 mol %, less than 3 mol %, less than 2 mol % degradation of the ascorbic acid initially present in the composition prior to storage. In certain embodiments, the composition is stored in a sealed container. In certain embodiments, the composition is stored in a multi-use container.

### Containers

In some embodiments, the high potency Vitamin C concentrate of the disclosure is administered with a second non-aqueous formulation (i.e., oil, ester and/or silicone carrier). The two compositions can be pre-filled into a “dual- chamber” container - a pump container in which two formulations are stored separately prior to dispense - with a high-potency Vitamin C concentrate of the invention in a first chamber, and a non-aqueous formulation in a second chamber. Some dual-chamber containers have two separate actuators/pumps, each having an orifice for dispensing one of the two formulations. Other dual-chamber containers contain two pumps and one actuator from which the two formulations are dispensed - either side-by-side (e.g., through two orifices), or from a single shared orifice. A non-limiting example of a dual-chamber container is described in U.S. Pat. No. 6,462,025.

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of ascorbic acid in a multi-use container which is pre-filled with a storage stable topical composition (e.g., as described herein).

Additional packaging for the container can be included. In some cases, the packaging provides a further barrier that prevents absorption of light and/or moisture from the atmosphere or surrounding environment.

### Methods of Preparation

Also provided by this disclosure are processes for stabilizing ascorbic acid for storage that include preparation of any one of the subject formulations (e.g., as described herein), e.g., by dissolving ascorbic acid in a non-aqueous solvent with a urea agent and chemical exfoliant components to provide a stable liquid composition capable of storage stability.

In some embodiments, the process includes combining:
1% to 20% by weight urea agent selected from urea, hydroxyethyl urea, and combination thereof;
10% to 94% by weight of a non-aqueous skin-compatible solvent comprising C(3-6)polyol, ethoxydiglycol, dimethyl ether, or a combination thereof;
2% to 50% by weight (e.g., such as 2 to 30%) of a chemical exfoliant; and
optionally one or more additional agents; with
5% to 28% by weight ascorbic acid;
thereby dissolving the ascorbic acid to produce storage stable, nonaqueous, single-phase clear liquid composition of ascorbic acid. In certain embodiments, the one or more additional agents are combined and include: 0.5% to 2% ferulic acid; and 0.5% to 2% pinus pinaster bark extract. In certain embodiments, the one or more additional agents are combined and include: 3% to 10% by weight azelaic acid.

In some embodiments, the process further includes: combining 0.5% to 2% by weight of Vitamin E and 1.5% to 5% by weight of an emulsifying agent to produce a second liquid composition; and combining the second liquid composition with the liquid composition of ascorbic acid to produce an emulsion. In some embodiments, the process further includes: combining 0.5% to 2% by weight of a lipid component and 1.5% to 5% by weight of an emulsifying agent to produce a second liquid composition; and combining the second liquid composition with the liquid composition of ascorbic acid to produce an emulsion.

Also provided are product storage stable formulations produced by the process according to any one of the embodiments described herein.

### DEFINITIONS

The following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

“At least one” means one or more, and also includes individual components as well as mixtures/combinations.

Numbers used in describing quantities of ingredients and/or reaction conditions are to be understood as being modified in all instances by the term “about.” Unless otherwise indicated, percentages and ratios are to be understood as based upon the total weight of the concentrate.

Numerical ranges are meant to include numbers within the recited range, and combinations of subranges between the given ranges. For example, a range from 1-5 includes 1, 2, 3, 4 and 5, as well as subranges such as 2-5, 3-5, 2-3, 2-4, 1-4, etc.

The terms “formulation” and “composition” are used interchangeably herein.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed. All patents and publications referred to herein are expressly incorporated by reference.

### Additional Embodiments

Additional Embodiments of this disclosure are described in the following aspects.

Aspect 1. A storage stable topical composition comprising:
a) 5% to 28% by weight ascorbic acid;
b) 5% to 20% by weight of a urea agent;
c) 2% to 30% by weight of a chemical exfoliant; and
d) less than 10% by weight in total of one or more optional additional components;
e) dissolved in a non-aqueous skin-compatible solvent comprising polyol, C₍₂₋₆₎ alkanediol, glycol ether, dimethyl ether, ethanol, isopropyl alcohol, or a combination thereof, wherein the ascorbic acid is dissolved at a concentration [AA] that is above its maximum concentration in the solvent alone [X], and the urea is dissolved at a concentration that is at least ([AA]-[X])*1.25.

Aspect 2. The composition of aspect 1, wherein the composition demonstrates less than 5 mol % degradation of the ascorbic acid after storage for 6 weeks at 40° C. ± 2° C. in a sealed container.

Aspect 3. The composition of aspect 1, wherein the composition demonstrates less than 2 mol % degradation of the ascorbic acid after storage for 6 months at 40° C. ± 2° C. in a multi-use container.

Aspect 4. The composition of aspect 1, wherein the composition demonstrates less than 5 mol % degradation of the ascorbic acid after storage for 12 months at 40° C. ± 2° C. in a multi-use container.

Aspect 5. The composition of aspect 1, wherein the urea agent is urea.

Aspect 6. The composition of aspect 1, wherein the urea agent is hydroxyethyl urea.

Aspect 7. The composition of aspect 1, wherein the urea agent comprises a mixture of urea and hydroxyethyl urea.

Aspect 8. The composition of any one of aspects 1-7, wherein the composition comprises 5-20% of the urea agent.

Aspect 9. The composition of any one of aspects 1-7, wherein the composition comprises 5-15% of the urea agent.

Aspect 10. The composition of any one of aspects 5-7, wherein the composition comprises 5-10% of the urea agent.

Aspect 11. The composition of any one of aspects 1-10, wherein the composition comprises 5-25% ascorbic acid.

Aspect 12. The composition of any one of aspects 1-10, wherein the composition comprises 5-25% ascorbic acid.

Aspect 13. The composition of aspects 1-10, wherein the composition comprises 20-25% ascorbic acid.

Aspect 14. The composition of any one of aspects 1-13, wherein the solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, ethanol, isopropyl alcohol, and dimethyl isosorbide.

Aspect 15. The composition of aspect 14, wherein the solvent is 1,3 propanediol.

Aspect 16. The composition of aspect 14, wherein the solvent is a mixture of 1,3 propanediol and 1,2 hexanediol.

Aspect 17. The composition of any one of aspects 1-16, wherein the chemical exfoliant is an alpha hydroxy acid or a benzoic acid.

Aspect 18. The composition of any one of aspects 1-17, wherein the chemical exfoliant is selected from glycolic acid, lactic acid, mandelic acid, salicylic acid, capryloyl salicylic acid, salicyloyl phytosphingosine, phenol, gluconolactone, lactobionic acid, maltobionic acid, and combinations thereof.

Aspect 19. The composition of aspect 17 or 18, wherein the chemical exfoliant is salicylic acid.

Aspect 20. The composition of aspect 19, wherein the composition comprises 2-20% by weight of salicylic acid.

Aspect 21. The composition of aspect 20, wherein the composition comprises 2% by weight of salicylic acid.

Aspect 22. The composition of aspect 20, wherein the composition comprises 5-15% by weight of salicylic acid.

Aspect 23. The composition of aspect 22, wherein the composition comprises 10% by weight of salicylic acid.

Aspect 24. The composition of any one of aspects 1-23, wherein the one or more optional additional components are selected from tocopherols, tocotrienols (e.g., alpha, beta, delta and gamma tocopherols or alpha, beta, delta and gamma tocotrienols), azelaic acid, cinnamic acid or cinnamic acid derivative, panthenol, pinus pinaster bark extract, emulsifying agent, hyaluronic acid complex, madecassoside, madecassoside asiaticoside, acetyl zingerone, bakuchiol, Diglycerin, bis ethylhexylhydroxydimethoxybenzylmalonate, and dimethyl isosorbide.

Aspect 25. The composition of any one of aspects 1-24, wherein the composition comprises:
25% by weight of ascorbic acid;
20% by weight of the urea agent; and
2% by weight of salicylic acid;
dissolved in 53% by weight of 1,3 propanediol.

Aspect 26. The composition of any one of aspects 1-24, wherein the composition comprises:
20% by weight of ascorbic acid;
17% by weight of the urea agent;
10% by weight of salicylic acid; and
53% by weight of 1,3 propanediol.

Aspect 27. The composition of any one of aspects 1-24, wherein the ratio of ascorbic acid to urea agent is 1.0 to 2.2.

Aspect 28. The composition of any one of aspects 1-24, wherein the ratio of ascorbic acid to urea agent is 1.10 to 1.25.

Aspect 29. The composition of aspect 24, wherein the one or more optional additional components comprise acetyl zingerone.

Aspect 30. The composition of aspect 29, wherein the composition comprises 2% or less by weight of the acetyl zingerone.

Aspect 31. The composition of aspect 30, wherein the composition comprises about 0.5% by weight of the acetyl zingerone.

Aspect 32. The composition of aspect 24, wherein the one or more optional additional components comprise a cinnamic acid or cinnamic acid derivative.

Aspect 33. The composition of aspect 32, wherein the cinnamic acid derivative is selected from ferulic acid, caffeic acid, coumaric acid, sinapinic acid, and derivatives thereof.

Aspect 34. The composition of aspect 33, wherein the composition comprises 0.1 to 2% by weight of the ferulic acid.

Aspect 35. The composition of aspect 33, wherein the composition comprises 1% or less by weight of the ferulic acid.

Aspect 36. The composition of aspect 33, wherein the composition comprises about 0.5% by weight of the ferulic acid.

Aspect 37. The composition of any one of aspects 1-36, wherein the composition comprises 40-60% by weight of the solvent comprising propanediol.

Aspect 38. The composition of any one of aspects 1-37, wherein the composition comprises 53% by weight of the solvent comprising propanediol.

Aspect 39. The composition of any one of aspects 1-37, wherein the composition comprises 43% by weigh of the solvent comprising propanediol.

Aspect 40. The composition of any one of aspects 1-37, wherein the composition comprises 43% by weigh of the solvent comprising propanediol and 10% of Dimethyl Isosorbide.

Aspect 41. The composition of aspect 24, wherein the one or more optional additional component comprises comprises 0.5% by weight of diglycerin and pinus pinaster bark extract.

Aspect 42. The composition of any one of aspects 1-23, wherein the one or more optional additional component comprises azelaic acid.

Aspect 43. The composition of aspect 42, wherein the composition comprises 3% to 10% by weight of the azelaic acid.

Aspect 44. The composition of aspect 42, wherein the composition comprises about 7.5% by weight of the azelaic acid.

Aspect 45. The composition of any one of aspects 1-23, wherein the one or more optional additional components comprises madecassoside.

Aspect 46. An emulsion composition, comprising:
the composition according to any one of aspects 1-45;
an oil component; and
an optional emulsifying agent.

Aspect 47. The emulsion composition of aspect 46, wherein the oil component is silicone-based.

Aspect 48. The emulsion composition of aspect 46 or 47, wherein the emulsion composition comprises an emulsifying agent.

Aspect 49. The emulsion composition of aspect 48, wherein the emulsifying agent is selected from polysorbates, laureth-4, potassium cetyl sulfate and silicone and silicone-elastomer-based emulsifiers and emulsifying blends.

Aspect 50. A ready-to-use topical preparation of a storage stable composition in a multi-use container which is pre-filled with the storage stable topical composition according to any one of aspects 1-45, wherein the multi-use container comprises means for dispensing a single dose of the storage stable topical composition.

Aspect 51. The preparation of aspect 50, wherein the storage stable topical composition demonstrates less than 5 mol % degradation of the ascorbic acid after storage for 6 weeks at 40° C. ± 2° C. in the container.

Aspect 52. The preparation of aspect 50, wherein the storage stable topical composition demonstrates less than 5 mol % degradation of the ascorbic acid after storage for 6 months at 40° C. ± 2° C. in the container.

Aspect 53. The preparation of any one of aspects 50-52, wherein the storage stable topical composition is sealed in the container.

Aspect 54. The preparation of any one of aspects 50-53, wherein the container is placed in packaging.

Aspect 55. A process for preparing a stable topical composition, the process comprising:
combining:
i) 5% to 28% by weight ascorbic acid
ii) 5% to 20% by weight of a urea agent;
iii) 2% to 30% by weight of a chemical exfoliant; and
iv) less than 10% by weight in total of one or more optional additional components; with
v) a non-aqueous skin-compatible solvent comprising polyol, C(2-6) alkanediol, glycol ether, dimethyl ether, ethanol, isopropyl alcohol, or a combination thereof, wherein the ascorbic acid is dissolved at a concentration [AA] that is above its maximum concentration in the solvent alone [X], and the urea is dissolved at a concentration that is at least ([AA]-[X])* 1.25.

Aspect 56. A product produced by the process according to aspect 55.

Aspect 57. The product of aspect 56, wherein the product is a chemical peeling formulation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

The invention is further defined by reference to the following examples. These examples are representative and should not be construed to limit the scope of the invention.

### EXAMPLES

### Example 1: Assessment of Formulation Components

A series of experiments were performed to assess and optimize the components of the subject formulations. AA refers to L-ascorbic acid. U refers to urea. % values are wt %.

### Summary of Experiments

### Ascorbic Acid to Urea

The maximum amount of AA solubilized in 1,3-propanediol before recrystallization was ~12%. This solubility limit was also observed for propylene glycol (1,2 propanediol).

First: completely solubilized AA/U in 1,3 propanediol at 20% AA, and 15% U.

Reduced to 10% U content, and still no recrystallization.

Reduced to 5% U content, and recrystallization occurred.

Tested 8% U content and recrystallization occurred.

10% U content thus appeared to be close to the minimum amount U required to solubilize 20% AA.

U in combination with a 15% AA content:

5% U prevented recrystallization

3.75% U prevented recrystallization

2.5% U resulted in recrystallization

Maximum saturation level experiments

30% AA, 20% U in 1,3 propanediol resulted in recrystallization

28% AA, 20% U resulted in fully solubilized AA with no recrystallization

The limitation of this composition is the solubility of U in 1,3 propanediol - ~27.8% saturation can be reached before recrystallization of U becomes apparent

Using these numbers is how the following equation was obtained for determining the amount of U, and thus the ratio of AA to U in high concentration ascorbic acid formulations: (AA-X)* 1.25 = U %, where X = the maximum % solubility of AA in the chosen solvent. In this case, X = 12%, as noted above.

The equation is relevant to compositions including a lower limit of 5% ascorbic acid because the inclusion of other polyols that provide very low or virtually no solubility of AA, such as dimethyl isosorbide (DMI). Therefore, when a mixture of propanediol and DMI is used as the solvent, for example, the X value can be 5% (maximum solubility of AA), depending on the ratio of propanediol and DMI used.

### Solvents

1,3 propanediol, 1,2 propanediol, butylene glycol, pentylene glycol, and hexanediol were identified as preferred solvents. 1,3 propanediol (trade name: Zemea) is inherently different from and preferable to the various polyols described. Below is a review of various polyols and reasons why 1,3 propanediol is unique and preferable:

1,3-propanediol, sometimes referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize ascorbic acid.

1,2-propanediol, otherwise referred to in the art as propylene glycol, although of low viscosity and possessing a moderate ability to solubilize ascorbic acid, is well-known for inducing skin irritation and sensitivity. Additionally, it is derived from petroleum and possesses an unpleasant odor, reminiscent of acetone.

1,3-butanediol, otherwise referred to in the art as butylene glycol, is of low viscosity, possesses a moderate ability to solubilize ascorbic acid, and is relatively gentle on skin. However, like propylene glycol, it is derived from petroleum (not environmentally friendly) and possesses an unpleasant odor, reminiscent of acetone.

Also applicable to dipropylene glycol.

1,5-pentanediol, otherwise referred to in the art as pentylene glycol, possesses a moderate ability to solubilize ascorbic acid, low odor, and certain versions are not derived from petroleum but from sugarcane or corn. However, upon application to skin, it imparts a “heavier”, less desirable texture on skin. Additionally, its recommended use level is capped at 5%, limiting usage as a primary solvent.

1,2-hexanediol possesses a moderate ability to solubilize ascorbic acid. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize ascorbic acid, are relatively gentle on skin, are low-odor, and are not derived from petroleum. However, they are of a very viscous nature, and impart not only an undesirable, “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize ascorbic acid and possesses a slight, but noticeable chemical odor reminiscent of chlorine.

### Urea Agents

Urea is preferable to hydroxyethyl urea. There are a number of reasons for this:

Urea, when used in sufficient low concentrations (10-15% and below) in leave-on applications, possesses desirable humectant, barrier-repairing and very mild keratolytic properties, which in combination are very effective at improving the feel and look of dry and/or rough skin.

Urea is naturally present not only in the human body but specifically in the skin, where it acts as a natural moisturizing factor (NMF).

Hydroxyethyl urea possesses similar humectant properties, but not the same level of barrier-repairing and mild keratolytic properties of urea.

Additionally, hydroxyethyl urea may contain trace amounts of diethanolamine, which is listed as a potential carcinogen by California’s Proposition 65, and requires a warning on products sold to consumers. For this reason, at least one manufacturer of hydroxyethyl urea has stated that it will discontinue production of this ingredient (AkzoNobel).

### Optional Additional Components

Additional ingredients were chosen for their compatibility with (e.g., miscibility in) 1,3 propanediol, 1,2 propanediol, and/or 1,3 butanediol. Additional notes and observations on each optional additional components which can be adapted for use in the compositions of this disclosure are shown below.

### Panthenol (pro-vitamin B5)

This is a humectant that shows soothing and moisturizing properties for skin. Both enantiomers, D-panthenol and L-panthenol, are potent humectants. However, only D-panthenol is converted into pantothenic acid in the skin, which confers additional benefits to skin (wound healing, for example).

Research shows that panthenol can reduce irritation to skin by other ingredients. Research also shows panthenol barrier-repairing ability (stimulation of physiologic lipid synthesis)

DL-panthenol is a racemic mixture of the two enantiomers; it is in powdered/crystal form. D-panthenol is a viscous liquid. DL-panthenol is freely soluble in 1,3 propanediol, 1,2 propanediol and 1,3 propanediol (up to 50%). D-panthenol is also freely soluble in 1,3 propanediol, 1,2 propanediol and 1,3 propanediol, with no risk of recrystallization at any concentration (as it is already liquid at room temperature).

Inhibition of transepidermal water loss is apparent at concentrations of 1% and above.

Panthenol is used as an optional additive in exemplary compositions of this disclosure.

### Hyaluronic Acid

Hyaluronic acid is a humectant that shows the ability to form a viscoelastic film on skin that prevents transepidermal water loss.

Hyaluronic acid is usually incorporated in aqueous solutions in its salt form, sodium hyaluronate. However, there is a raw material blend that is largely free from water, in which it is incorporated in a vehicle of glyceryl polymethacrylate, butylene glycol (1,3 butanediol), and natto gum (trade name Hydrafilm 3MW by The Innovation Company). This makes hyaluronic acid compatible with the nonaqueous formulations of the present disclosure.

Documents from The Innovation Company show usage of this material up to 9.1% by weight of the final formula.

The chemical composition is as follows:
75-85% glyceryl polymethacrylate,
15-20% butylene glycol,
0.5-2% natto gum, and
0.5-2% hyaluronic acid.

Hyaluronic acid is optionally added to exemplary compositions of this disclosure. In some cases, 0.5-2.0% by weight of hyaluronic acid is utilized.

### Pinus Pinaster Bark Extract

Components of the bark extract of pinus pinaster species show the ability to recycle vitamin C.

Additionally, there is research to show general antioxidant, anti-inflammatory and anti-acne properties of Pinus Pinaster bark extract.

Pycnogenol may be used as an alternative when pinus pinaster bark extract is desired.

A material blend from Kinetik called Pantrofina Skin360 (PS360) is optionally utilized in the subject formulations.

PS360, unlike pycnogenol, is already in liquid form as it uses diglycerin as a solvent, making it very easy to incorporate into the compositions of this disclosure.

Additionally, Res Pharma Industriale provides in-vitro and clinical data to show effectiveness against free radical damage, inflammation and acne at a concentration of 0.5% by weight of PS360.

The chemical composition is as follows:
90-95% diglycerin,
5-10% pinus pinaster bark extract.

Pinus Pinaster bark extract is optionally added to exemplary compositions of this disclosure.

### Madecassoside

Centella Asiatica extract can be used for its skin soothing properties.

Madecassoside is a highly purified glycosylated triterpene of Centella Asiatica. It is sold by raw material supplier SEPPIC, who share in-vitro and clinical data showing its anti-inflammatory and other effects on skin.

This is a very expensive ingredient ($6.10 per gram), but clinical data from SEPPIC shows desirable ability to reduce erythema (skin redness) in concentrations of 0.2%.

At a concentration of 0.2%, madecassoside is soluble in 1,3 propanediol, 1,2 propanediol and 1,3 butanediol.

Centella Asiatica extract or Madecassoside is optionally added to exemplary compositions of this disclosure.

In some embodiments, madecassoside is madecassoside asiaticoside.

### Azelaic Acid

Azelaic acid (AzA) is well studied for its ability to treat acne, rosacea and melasma, due to the fact that it was studied and sold as a prescription drug. Though poorly understood, these effects are believed to be a result of AzA’s anti-bacterial, anti-inflammatory, and keratolytic effects, as well as its unique ability to cause apoptosis in abnormal melanocytes.

It is very poorly soluble in most solvents. As a result, all products currently on the market, both prescription and cosmetic, are sold as opaque emulsions, where the AzA is not solubilized but instead finely milled into a powder and suspended in the viscous vehicle.

Because of an inability to solubilize AzA, a preferred component for maximizing delivery into the skin of active ingredients, the team behind prescription product Finacea (currently considered to be the gold standard) chose to manipulate pH, as they discovered that, counterintuitively, a salt form of AzA (formed in aqueous environments in which the pH is higher than the pKa of AzA, 4.15), is slightly better at penetrating skin.

I’ve discovered that AzA can be solubilized in 1,3 propanediol at relatively high concentrations - up to 10%.

The solubility of AzA in 1,3 propanediol can be slightly increased by the presence of hydroxyethyl urea.

For example, it is possible to solubilize 7.5% AzA with 10% Ascorbic Acid (AA), 5% Urea in a 1,3 propanediol solvent base.

Azelaic acid is optionally added to exemplary compositions of this disclosure.

### Ferulic Acid

Ferulic acid is an antioxidant that increases AA’s photoprotective effect on skin. It can also somewhat stabilize AA in aqueous systems.

Ferulic acid is readily soluble in 1,3 propanediol, 1,2 propanediol, 1,3 butanediol and dimethyl isosorbide.

Isosorbide can in some cases increase the effectiveness of ferulic acid by enhancing skin penetration.

Ferulic acid is optionally added to exemplary compositions of this disclosure.

### Acetyl Zingerone

Acetyl zingerone is a broad-spectrum antioxidant that can prevent lipid peroxidation. It was engineered to be a more stable, more potent derivative of zingerone.

Sytheon provides in-vitro and clinical data showing its antioxidant, photoprotective, and anti-aging properties.

Acetyl zingerone may be used as a replacement for tocopherol.

Acetyl zingerone is readily soluble in 1,3 propanediol, 1,2 propanediol and 1,3 butanediol at the desired concentrations (0.5-1%), eliminating the need for emulsifiers as would be required for tocopherol.

Acetyl zingerone is optionally added to exemplary compositions of this disclosure.

### Glycyrrhizic Acid

Glycyrrhizic acid, like many other derivatives from licorice root (Glycyrrhiza Glabra, Glycyrrhiza Uralensis), shows anti-inflammatory, antioxidant and skin lightening properties.

Unlike 18B-glycyrrhetinic acid, glycyrrhizic acid shows solubility in 1,3-propanediol.

Other derivatives of licorice root can be use, such as dipotassium glycyrrhizate, monoammonium glycyrrhizate, etc.

Glycyrrhizic acid is optionally added to exemplary compositions of this disclosure.

### Example 2: Exemplary Formulations

### Ratio of Ascorbic Acid to Urea

In order to determine a desirable ratio of ascorbic acid to urea for the compositions of this disclosure, the maximum concentration for ascorbic acid that can be solubilized is first determined, with heat exposure (not exceeding 80oC in order to prevent degradation of ascorbic acid), in a given solvent without precipitation upon cooling. Experiments revealed this concentration to be approximately 10-12% for 1,3 propanediol, propylene glycol (1,2 propanediol) and butylene glycol (1,3 butanediol), and significantly lower for dimethyl isosorbide.

Next, concentrations of ascorbic acid beyond the aforementioned maximum concentration are solubilized, using urea as a co-solvent. Repeated experiments of this nature, using differing concentrations and ratios of urea to ascorbic acid, revealed the following relationship between these two substances (ascorbic acid and urea) that is useful to create fully solubilized composition which is storage stable: (AA-X)^{∗}1.25 = U
AA = concentration of ascorbic acid
X = maximum solubilization point of ascorbic acid in solvent of choice
U = concentration of urea

Compositions having an ascorbic acid concentration as low as 5% can be prepared in cases where the polyol solvents used provide very low solubility, such as dimethyl isosorbide (DMI). Therefore, a mixture of propanediol and DMI, for example, can yield an X value of 5% (maximum solubility of AA), depending on the ratio of propanediol and DMI.

In general, 1,3 propanediol is preferred over 1,2 propanediol, butylene glycol, pentylene glycol, or hexanediol. 1,3 propanediol is preferable to various polyols described in the art. Below is a review of various polyols and reasons why 1,3 propanediol is unique and preferable:

1,3 propanediol, otherwise referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize ascorbic acid.

1,2 propanediol, otherwise referred to in the art as propylene glycol, although of low viscosity and possessing a moderate ability to solubilize ascorbic acid, induces skin irritation and sensitivity. Additionally, it is derived from petroleum and possesses an unpleasant odor, reminiscent of acetone.

1,3 butanediol, otherwise referred to in the art as butylene glycol, is of low viscosity, possesses a moderate ability to solubilize ascorbic acid, and is relatively gentle on skin. However, like propylene glycol, it is derived from petroleum (not environmentally friendly) and possesses an unpleasant odor, reminiscent of acetone.

Note that these properties also apply to dipropylene glycol.

1,5 pentanediol, otherwise referred to in the art as pentylene glycol, possesses a moderate ability to solubilize ascorbic acid, low odor, and certain versions are not derived from petroleum but from sugarcane or corn. However, upon application to skin, it imparts a “heavier”, less desirable texture on skin. Additionally, its recommended use level is generally capped at 5%, limiting usage as a primary solvent.

1,2 hexanediol possesses a moderate ability to solubilize ascorbic acid. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize ascorbic acid, are relatively gentle on skin, are low-odor, and are not derived from petroleum. However, they are highly viscous, and impart not only an undesirable “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize ascorbic acid and possesses a slight, but noticeable chemical odor reminiscent of chlorine.

Urea is preferable to hydroxyethyl urea. There are a number of reasons for this, as summarized below:

Urea, when used in sufficient low concentrations (10-15% and below) in leave-on applications, possesses desirable humectant, barrier-repairing and very mild keratolytic properties, which in combination are very effective at improving the feel and look of dry and/or rough skin. Urea is naturally present not only in the human body but specifically in the skin, where it acts as a natural moisturizing factor (NMF).

Hydroxyethyl urea possesses similar humectant properties, but not the barrier-repairing and mild keratolytic properties of urea. Additionally, hydroxyethyl urea may contain trace amounts of diethanolamine, a potential carcinogen.

Additional ingredients can be included which are compatible with the ascorbic acid / solvent / urea combination of interest.

The exemplary formulations of Table 2 were prepared and assessed as having desirable properties including storage stability.

TABLE 2

Components of Exemplary Compositions (% by weight)

| | | | | |
|---|---|---|---|---|
| Formulation | 1 | 2 | 3 | 4 |
| ascorbic acid | 25% | 20% | 20% | 5% |
| urea / hydroxyethyl | 20% | 17% | 17% | 5% |
| urea | | | | |
| solvent | 53% 1,3-propanediol | 53% 1,3-propanediol (and) dimethyl isosorbide | 43% 1,3-propanediol | 50% 1,3-propanediol |
| chemical exfoliant | 2% salicylic acid | 10% salicylic acid | 10% salicylic acid | 20% salicylic acid |
| Additive | | | 10% Dimethyl Isosorbide | 20% Dimethyl Isosorbide |

### Example 3: Storage Stability Studies

### Stability Method

Samples are stored in sealed containers at 40° C. for up to 12 weeks. Preliminary results at 6 weeks are shown in Table 3. In general, 6 weeks storage under these conditions is expected to be equivalent to storage for 1 year at room temperature. The compositions in the containers are sampled every week, and assessed for levels of degradation of vitamin C using HPLC analysis.

### Compositions

Exemplary compositions were prepared containing either approx. 20% vitamin C (Formulations 2-3 referred to in Table 2) or approx. 25% vitamin C (Formulation 1 referred to in Table 2).

The storage stability of these compositions was compared to control compositions that included the same amount of vitamin C dissolved in water with no additional ingredients. The results are shown in Table 3. The exemplary the exemplary mask (approx. 25% vitamin C) composition is still within specification after 6 weeks, as opposed to the control compositions which fell out of specification (OOS) by week 3 of testing (or equivalent to 6 months at room temperature).

TABLE 3

Storage stability

| Storage time | | % vitamin C by HPLC | |
|---|---|---|---|
| Week 40° C. | Equiv. Months RT | Mask | Mask Control |
| 0 | 0 | 26.25 | 25.65 |
| 1 | 2 | 26.52 | 24.67 |
| 2 | 4 | 26.81 | 23.83 |
| 3 | 6 | 25.59 | 19.39* |
| 4 | 8 | 24.40 | 17.32* |
| 5 | 10 | 22.66 | 15.56* |
| 6 | 12 | 22.56 | 13.83* |

^{∗} indicates the samples were assessed as being OOS according to Out of Specification (OOS) Standards: Serum (20% vitamin C) 18.00 % wt or less (+2.00 margin of error); and Mask (25% vitamin C) 22.50 % wt or less (+2.50 margin of error).

### Example 4: Comparative Studies

U.S. Patent No. 6,020,367 (patent ‘367) attempted to show the viability of “supersaturated solutions” of vitamin C in a polyol. Several compositions of patent ‘367 were prepared in accordance with the disclosure, However, many of the “supersaturated solutions” of vitamin C patent ‘367 do not actually remain solubilized at room temperature over time. Rather, the solutions lead to development of vitamin C crystals which at first create a cloudy appearance and then settle downward. Such compositions are non-uniform and unsuitable for use as end products.

### Glycerin Solvent

A mixture of 25% ascorbic acid and 75% glycerin was prepared. The ascorbic acid was and solubilized with heating at 95° C. to produce a transparent solution. Upon cooling to room temperature, crystallization became apparent within the first 24 hours of storage.

### Butylene Glycol Solvent

According to patent ‘367 butylene glycol has a lower ability to solubilize ascorbic acid.

A mixture of 25% ascorbic acid and 75% butylene glycol was prepared. Even with heating at the maximum temperature of 95° C. (under agitation), butylene glycol failed to solubilize the ascorbic acid content, leaving a “cloudy” appearance and sedimentation upon cessation of agitation.

### Propylene Glycol Solvent

According to patent ‘367 propylene glycol has the lowest ability of these solvents to solubilize ascorbic acid. A mixture of 25% ascorbic acid and 75% propylene glycol was prepared. The ascorbic acid was and solubilized with heating at 95° C. to produce a transparent solution. Upon cooling to room temperature, crystallization became apparent within the first 24 hours of storage.

It is important to note the fragile nature of ascorbic acid renders it sensitive not only to the presence of water and air, but also heat. When heated above 80° C., even in anhydrous vehicles such as polyols, there is a risk for degradation of the ascorbic acid. The solutions described above prepared according to the direction of patent ‘367, when heated to the described range of 85-95° C., showed signs of degradation.

U.S. Publication No. 2007/0077261 (publication ‘261) discloses compositions including broad ranges of ascorbic acid and urea, but fails to identify both the “floor” (minimum amount of urea required to solubilize a certain amount of ascorbic acid) and the “ceiling” (maximum amount of ascorbic acid that can be solubilized through this method).

Example 3 of publication ‘261 discloses a composition including: 50% propylene glycol, 22% urea and 28% ascorbic acid, heated to 75° C. with agitation until transparent, then cooled to room temperature. This example was reproduced. The solution started to precipitate within 24 hours, demonstrating a failure to understand and elucidate the required ratio of urea to ascorbic acid.

Using the equation of this disclosure set forth above, the correct concentration of urea to solubilize 28% ascorbic acid in propylene glycol would be 20% (the proper “floor”). Indeed, a solution of 28% ascorbic acid and 20% urea in propylene glycol was prepared and remained fully solubilized even after 30 days of storage at room temperature. Furthermore, experiments reveal that these concentrations of ascorbic acid (28%) and urea (20%), also represent the maximum concentrations soluble in propylene glycol, butylene glycol and propanediol, before urea itself starts to precipitate in solution (the “ceiling”).

Experiments showed that no concentration of urea within the 5-40% range can solubilize 40% ascorbic acid in a polyol base:
40% ascorbic acid, 5% urea, 55% propylene glycol
40% ascorbic acid, 10% urea, 50% propylene glycol
40% ascorbic acid, 20% urea, 40% propylene glycol
40% ascorbic acid, 40% urea, 20% propylene glycol

All mixtures were heated to 85° C. However, none were solubilized even after agitation at maximum temperature of 85° C.

In addition, the urea content disclosed in several examples of publication ‘261 is not only unnecessarily high (likely because of a failure to identify the “floor”), but also renders the compositions unusable as leave-on facial products. These compositions, when applied to the face, produce an intense burning and stinging sensation that is immediately apparent. This is likely due to urea’s keratolytic properties. In leave-on products intended for the face, maximum urea content is usually 10-15%. Formulation 5 of Table 2 is identified as a rinse-off product.

While the illustrative embodiments of the invention have been described with particularity, it will be understood that various other modifications will be apparent to, and can be readily made by those skilled in the art, without departing from the spirit and scope of the invention. Accordingly, it is not intended that the scope of the claims appended hereto be limited to the examples and descriptions set forth hereinabove but rather that the claims be construed as encompassing all the features of patentable novelty in the present invention, including all features which would be treated as equivalents by persons having ordinary skill in the art of formulating topically-applied personal care and dermatological products.

What is claimed is:

1. A storage stable topical composition comprising:
a. 5% to 28% by weight ascorbic acid;
b. 5% to 20% by weight of a urea agent;
c. 2% to 30% by weight of a chemical exfoliant; and
d. less than 10% by weight in total of one or more optional additional components;
e. dissolved in a non-aqueous skin-compatible solvent comprising polyol, C(₂-₆) alkanediol, glycol ether, dimethyl ether, ethanol, isopropyl alcohol, or a combination thereof, wherein the ascorbic acid is dissolved at a concentration [AA] that is above its maximum concentration in the solvent alone [X], and the urea is dissolved at a concentration that is at least ([AA]-[X])*1.25.

2. The composition of claim 1, wherein the composition demonstrates less than 5 mol % degradation of the ascorbic acid after storage for 6 weeks at 40° C. ± 2° C. in a sealed container.

3. The composition of claim 1, wherein the composition demonstrates less than 2 mol % degradation of the ascorbic acid after storage for 6 months at 40° C. ± 2° C. in a multi-use container.

4. The composition of claim 1, wherein the composition demonstrates less than 5 mol % degradation of the ascorbic acid after storage for 12 months at 40° C. ± 2° C. in a multi-use container.

5. The composition of claim 1, wherein the urea agent is urea.

6. The composition of claim 1, wherein the urea agent is hydroxyethyl urea.

7. The composition of claim 1, wherein the urea agent comprises a mixture of urea and hydroxyethyl urea.

8. The composition of any one of claims 1-7, wherein the composition comprises 5-20% of the urea agent.

9. The composition of any one of claims 1-7, wherein the composition comprises 5-15% of the urea agent.

10. The composition of any one of claims 5-7, wherein the composition comprises 5-10% of the urea agent.

11. The composition of any one of claims 1-10, wherein the composition comprises 5-25% ascorbic acid.

12. The composition of any one of claims 1-10, wherein the composition comprises 5-25% ascorbic acid.

13. The composition of claims 1-10, wherein the composition comprises 20-25% ascorbic acid.

14. The composition of any one of claims 1-13, wherein the solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, ethanol, isopropyl alcohol, and dimethyl isosorbide.

15. The composition of claim 14, wherein the solvent is 1,3 propanediol.

16. The composition of claim 14, wherein the solvent is a mixture of 1,3 propanediol and 1,2 hexanediol.

17. The composition of any one of claims 1-16, wherein the chemical exfoliant is an alpha hydroxy acid or a benzoic acid.

18. The composition of any one of claims 1-17, wherein the chemical exfoliant is selected from glycolic acid, lactic acid, mandelic acid, salicylic acid, capryloyl salicylic acid, salicyloyl phytosphingosine, phenol, gluconolactone, lactobionic acid, maltobionic acid, and combinations thereof.

19. The composition of claim 17 or 18, wherein the chemical exfoliant is salicylic acid.

20. The composition of claim 19, wherein the composition comprises 2-20% by weight of salicylic acid.

21. The composition of claim 20, wherein the composition comprises 2% by weight of salicylic acid.

22. The composition of claim 20, wherein the composition comprises 5-15% by weight of salicylic acid.

23. The composition of claim 22, wherein the composition comprises 10% by weight of salicylic acid.

24. The composition of any one of claims 1-23, wherein the one or more optional additional components are selected from tocopherols, tocotrienols (e.g., alpha, beta, delta and gamma tocopherols or alpha, beta, delta and gamma tocotrienols), azelaic acid, cinnamic acid or cinnamic acid derivative, panthenol, pinus pinaster bark extract, emulsifying agent, hyaluronic acid complex, madecassoside, madecassoside asiaticoside,acetyl zingerone, bakuchiol, Diglycerin, bis ethylhexylhydroxydimethoxybenzylmalonate, and dimethyl isosorbide.

25. The composition of any one of claims 1-24, wherein the composition comprises:
25% by weight of ascorbic acid;
20% by weight of the urea agent; and
2% by weight of salicylic acid;
dissolved in 53% by weight of 1,3 propanediol.

26. The composition of any one of claims 1-24, wherein the composition comprises:
20% by weight of ascorbic acid;
17% by weight of the urea agent; and
10% by weight of salicylic acid;
53% by weight of 1,3 propanediol.

27. The composition of any one of claims 1-24, wherein the ratio of ascorbic acid to urea agent is 1.0 to 2.2.

28. The composition of any one of claims 1-24, wherein the ratio of ascorbic acid to urea agent is 1.10 to 1.25.

29. The composition of claim 24, wherein the one or more optional additional components comprise acetyl zingerone.

30. The composition of claim 29, wherein the composition comprises 2% or less by weight of the acetyl zingerone.

31. The composition of claim 30, wherein the composition comprises about 0.5% by weight of the acetyl zingerone.

32. The composition of claim 24, wherein the one or more optional additional components comprise a cinnamic acid or cinnamic acid derivative.

33. The composition of claim 32, wherein the cinnamic acid derivative is selected from ferulic acid, caffeic acid, coumaric acid, sinapinic acid, and derivatives thereof.

34. The composition of claim 33, wherein the composition comprises 0.1 to 2% by weight of the ferulic acid.

35. The composition of claim 33, wherein the composition comprises 1% or less by weight of the ferulic acid.

36. The composition of claim 33, wherein the composition comprises about 0.5% by weight of the ferulic acid.

37. The composition of any one of claims 1-36, wherein the composition comprises 40-60% by weight of the solvent comprising propanediol.

38. The composition of any one of claims 1-37, wherein the composition comprises 53% by weight of the solvent comprising propanediol.

39. The composition of any one of claims 1-37, wherein the composition comprises 43% by weigh of the solvent comprising propanediol.

40. The composition of any one of claims 1-37, wherein the composition comprises 43% by weigh of the solvent comprising propanediol and 10% of Dimethyl Isosorbide.

41. The composition of claim 24, wherein the one or more optional additional component comprises comprises 0.5% by weight of diglycerin and pinus pinaster bark extract.

42. The composition of any one of claims 1-23, wherein the one or more optional additional component comprises azelaic acid.

43. The composition of claim 42, wherein the composition comprises 3% to 10% by weight of the azelaic acid.

44. The composition of claim 42, wherein the composition comprises about 7.5% by weight of the azelaic acid.

45. The composition of any one of claims 1-23, wherein the one or more optional additional components comprises madecassoside.

46. An emulsion composition, comprising:
the composition according to any one of claims 1-45;
an oil component; and
an optional emulsifying agent.

47. The emulsion composition of claim 46, wherein the oil component is silicone-based.

48. The emulsion composition of claim 46 or 47, wherein the emulsion composition comprises an emulsifying agent.

49. The emulsion composition of claim 48, wherein the emulsifying agent is selected from polysorbates, laureth-4, potassium cetyl sulfate and silicone and silicone-elastomer-based emulsifiers and emulsifying blends.

50. A ready-to-use topical preparation of a storage stable composition in a multi-use container which is pre-filled with the storage stable topical composition according to any one of claims 1-44, wherein the multi-use container comprises means for dispensing a single dose of the storage stable topical composition.

51. The preparation of claim 50, wherein the storage stable topical composition demonstrates less than 5 mol % degradation of the ascorbic acid after storage for 6 weeks at 40° C. ± 2° C. in the container.

52. The preparation of claim 51, wherein the storage stable topical composition demonstrates less than 5 mol % degradation of the ascorbic acid after storage for 6 months at 40° C. ± 2° C. in the container.

53. The preparation of any one of claims 50-52, wherein the storage stable topical composition is sealed in the container.

54. The preparation of any one of claims 50-53, wherein the container is placed in packaging.

55. A process for preparing a stable topical composition, the process comprising:
combining:
i. 5% to 28% by weight ascorbic acid
ii. 5% to 20% by weight of a urea agent;
iii. 2% to 30% by weight of a chemical exfoliant; and
iv. less than 10% by weight in total of one or more optional additional components; with
v. a non-aqueous skin-compatible solvent comprising polyol, C(2-6) alkanediol, glycol ether, dimethyl ether, ethanol, isopropyl alcohol, or a combination thereof, wherein the ascorbic acid is dissolved at a concentration [AA] that is above its maximum concentration in the solvent alone [X], and the urea is dissolved at a concentration that is at least ([AA]-[X])* 1.25.

56. A product produced by the process according to claim 55.

57. The product of claim 56, wherein the product is a chemical peeling formulation.
